(19) United States
(12) Patent Application Publication
(10) Pub. No.: US 2022/0304664 A1
(43) Pub. Date: Sep. 29, 2022

(54) APPARATUS AND METHODS FOR BODILY FLUID SAMPLE COLLECTION

(71) Applicant: Jeremy Hammer, Portland, OR (US)

(72) Inventor: Jeremy Hammer, Portland, OR (US)

(21) Appl. No.: 17/213,899

(22) Filed: Mar. 26, 2021

(51) Int. Cl. A61B 10/00

(52) U.S. Cl. CPC A61B 10/0045; A61B 5/150251

(57) ABSTRACT

A system acquiring body fluid samples is provided. One embodiment comprises a housing with a sampling channel extending therethrough and defined by an inlet port and an outlet port, wherein the inlet port secures a needle that penetrates a patient; a diversion chamber body portion directly secured to the housing with at least one diversion chamber, wherein the diversion chamber is in fluid communication with the sampling channel, wherein an initial first portion of the body fluid received from the needle is transferred through the sampling channel inlet port and into the diversion chamber, and wherein a subsequent second portion of the body fluid received from the needle is communicated through the sampling channel to the outlet port for collection into a collection device while the initially received first portion of the body fluid is retained in the diversion chamber.

### BACKGROUND OF THE INVENTION

In the arts of collecting bodily fluid samples, such as blood or the like, collected samples may become contaminated during the sample collection process. Typically, a primary blood collection device, commonly known as a needle, may have a contaminant thereon prior to initiation of the collection process. When the needle is inserted into the patient (used to puncture the patient), any present contaminant on the needle may be drawn into the needle during the subsequent bodily fluid collection process. Further, other tissue and or fluids (contaminants) may be acquired in the needle tip at the practitioner is pushing the needle tip to the sampling location. Accordingly, the contaminant and the collected bodily fluid become intermingled.

During the testing phase, present contaminants may result in erroneous test results. For example, blood cultures are a standard test to detect microbial pathogens in a patient's blood. Other bodily fluid samples are used to diagnose infectious diseases, autoimmune disorders, tumors, and other potential diseases and disorders. Any present contamination in a collected bodily fluid sample can lead to false positive bodily-fluid samples by introducing epidermal bacterium and/or other contaminants through the needle puncture process. For example, Cerebrospinal Fluid Analysis can result in a false positive reading of phagocytosis of erythrocytes due to blood contamination.

Accordingly, in the arts of collecting bodily fluid samples, there is a need in the arts for improved methods, apparatus, and systems for acquiring contaminant free bodily fluid samples.

### SUMMARY OF THE INVENTION

Embodiments for acquiring body fluid samples is provided. One embodiment comprises a housing with a sampling channel extending therethrough and defined by an inlet port and an outlet port, wherein the inlet port secures a needle that penetrates a patient; a diversion chamber body portion directly secured to the housing with at least one diversion chamber, wherein the diversion chamber is in fluid communication with the sampling channel, wherein an initial first portion of the body fluid received from the needle is transferred through the sampling channel inlet port and into the diversion chamber, and wherein a subsequent second portion of the body fluid received from the needle is communicated through the sampling channel to the outlet port for collection into a collection device while the initially received first portion of the body fluid is retained in the diversion chamber.

### BRIEF DESCRIPTION OF THE DRAWINGS

The components in the drawings are not necessarily to scale relative to each other. Like reference numerals designate corresponding parts throughout the several views.

FIG. 1 is an isometric view of an example embodiment of a bodily-fluid diversion device.

FIG. 2 is a cross-section view of an example embodiment of the bodily-fluid diversion device.

FIG. 3 is an isometric view of an alternative embodiment of a bodily-fluid diversion device.

FIG. 4 is a block diagram of an alternative embodiment of the bodily-fluid diversion device.

FIG. 5 is a cross-section view of an example embodiment of the bodily-fluid diversion device.

FIG. 6 is an isometric view of an alternative embodiment of the bodily-fluid diversion device.

FIG. 7 is a block diagram of an alternative embodiment of the bodily-fluid diversion device.

FIG. 8 is an isometric view of an alternative embodiment of the bodily-fluid diversion device.

FIG. 9 is a block diagram of an alternative embodiment of the bodily-fluid diversion device.

FIG. 10 is a cross-section view of an example alternative embodiment showing various optional features of the bodily-fluid diversion device.

FIG. 11 is an isometric view of an example embodiment of a bodily-fluid diversion device with an air tank.

FIG. 12 is a cross-section view of the example embodiment of the bodily-fluid diversion device with the air tank.

FIG. 13 is a block diagram of an alternative embodiment of the bodily-fluid diversion device with an air tank.

FIG. 14 is a cross-section view of the example embodiment of a bodily-fluid diversion device with the air tank.

FIG. 15 is a cross-section view of an example embodiment of a bodily-fluid diversion device with an air tank and no diversion chamber cap.

FIG. 16 is a cross-section view of an example embodiment of a bodily-fluid diversion device with an air tank and a filter at a distal end of the diversion chamber.

FIG. 17 is a cross-section view of an example embodiment of a bodily-fluid diversion device with an air tank having an air outlet.

### DETAILED DESCRIPTION

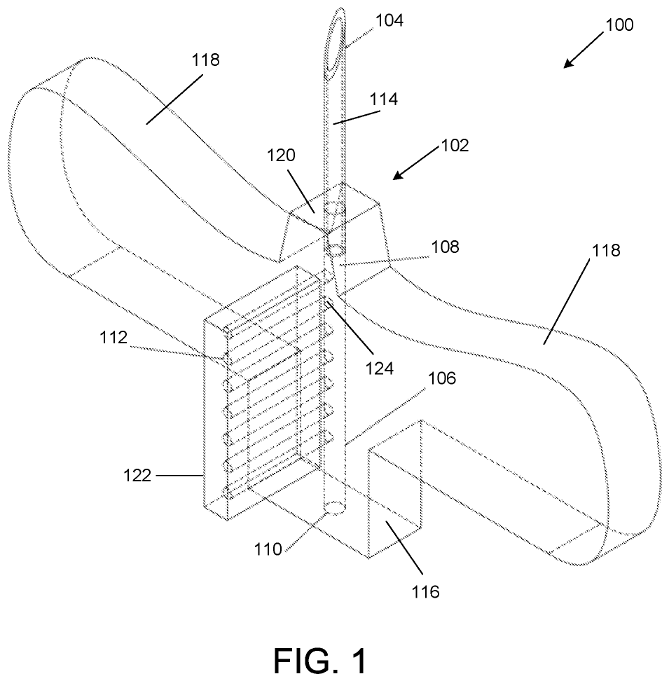

FIG. 1 is an isometric view of an example embodiment of a bodily-fluid diversion device 100. Bodily-fluid diversion device 100 embodiments are described that reduce or eliminate false positive readings in blood cultures and other bodily-fluid samples by reducing contamination from undesired contamination, such as, but not limited to, bacteria, portions of bodily-fluids and/or bodily tissues, organs, bones, and the like.

The bodily-fluid diversion device 100 may comprise a housing 102 that optionally secures a needle 104. The needle 104 is a device configured to penetrate (puncture) the patient's body to obtain a sample of bodily fluids. Preferably, the needle 104 is hollow with a channel therethrough to transport the acquired body fluid sample, and is in fluid communication with the housing 102. In other embodiments, the inlet port 108 may be fluidly coupled to the proximal end of the needle 104 via a tube or the like.

The housing 102 comprises a sampling channel 106, an inlet port 108, an outlet port 110, and one or optionally more diversion chambers 112. Alternatively, the diversion chambers 112 may be disposed in a diversion chamber body portion 122 that is directly secured to the housing 102. A proximal end of the needle 104 is secured at the inlet port 108 of the housing 102. In the various embodiments, an initially received portion of the body fluid sample, potentially with the undesired contaminants, are diverted into the diversion chambers 112 as the body fluid sample is transported through the sampling channel 106 towards the outlet port 110.

As the diversion chambers 112 are filled with the first portion of the acquired bodily fluid sample, the force of the capillary action in an example embodiment will draw in and retain the first portion of bodily fluid sample within the diversion chambers 112. Prior to use, the diversion chambers 112 are typically filled with air at atmospheric pressure. The fluid pressure of the incoming first portion of the body fluid sample is greater that the air pressure within the diversion chambers 112. As the first portion of the body fluid sample flows into the diversion chambers 112, the air is compressed and the air pressure increases. This compressed higher-pressure air retards flow of the initial first portion of the body fluid into the diversion chamber 112. At some point the increased air pressure may reach the fluid pressure and may then halt the flow of the initial first portion of the body fluid into the diversion chamber 112. At this juncture, after the diversion chambers 112 have been filled, the second portion of the body fluid sample can be collected since the fluid pressure of the first portion of the body fluid sample residing in the diversion chambers 112 will be the same as the fluid pressure of the incoming second portion of the body fluid sample. The size and dimensions of the diversion chambers 112 may be designed to acquire a desired amount of the initial first portion of the body fluid. Further, the size and dimensions of the diversion chambers 112 in proximity to the junction 124 can be designed so that flow of the second portion of the body fluid sample through the sampling channel 106 remains laminar, wherein the laminar flow does not disturb, and thus facilitates retention of, the first portion of the body fluid sample in the diversion chambers 112.

After the first portion of the body fluid sample is sequestered in the diversion chambers 112, the subsequently acquired second portion of body fluid sample will bypass the diversion chambers 112 retaining the first portion of bodily fluid, thereby allowing for the subsequent second portion of body fluid sample to flow from the inlet port 108, through the sampling channel 106, and to the outlet port 110. Accordingly, the second portion of the body fluid sample can be acquired without being contaminated with any contaminants that might be in the first portion of body fluid sample that has been retained within the diversion chambers 112.

The disclosed systems and methods for embodiments of a bodily-fluid diversion device 100 will become better understood through review of the following detailed description in conjunction with the figures. The detailed description and figures provide examples of the various inventions described herein. Those skilled in the art will understand that the disclosed examples may be varied, modified, and altered without departing from the scope of the inventions described herein. Many variations are contemplated for different applications and design considerations, however, for the sake of brevity, each and every contemplated variation is not individually described in the following detailed description.

Throughout the following detailed description, a variety of examples for systems and methods for a bodily-fluid diversion device 100 are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional, elements or method steps not expressly recited.

Terms such as “first”, “second”, and “third” are used to distinguish or identify various members of a group, or the like, and are not intended to denote a serial, chronological, or numerical limitation.

“Coupled” means connected, either permanently or releasably, whether directly or indirectly through intervening components.

“Communicatively coupled” means that an electronic device exchanges information with another electronic device, either wirelessly or with a wire based connector, whether directly or indirectly through a communication network. “Controllably coupled” means that an electronic device controls operation of another electronic device.

Returning to FIG. 1, the inlet port 108 secures the proximate end of the needle 104 to the housing 102. The inlet port 108 receives the body fluid sample from the needle 104. In practice, the needle 104 is forced into a particular area of the patient by a practitioner, such as a doctor, nurse or the like. When the needle 104 punctures the patient's body, the practitioner typically directs the needle 104 into a particular portion of the patient that has the body fluid sample of interest. For example, if the intended body fluid sample is blood, the practitioner direct the needle 104 into a vein of the patient. As another non-limiting example, if the intended body fluid sample of interest is spinal fluid, the practitioner directs the needle into the patient's spine.

One skilled in the art appreciates that contaminants may initially enter into the distal end of the needle 104. Contaminants may have been on the surface or in the needle channel 114 prior to puncturing the patient, and may enter into the channel 114 of the needle 104 when the needle punctures the patient. Additionally, or alternatively, some other body fluids and/or tissue that are not intended to be sampled may initially enter into the channel 114 of the needle 104 during the puncture process as the needle is being directed into the portion of interest where the body fluid sample is to be acquired. Such undesirable acquired body fluids and/or tissue are considered as a contaminant herein since this bodily fluid and/or tissue is different from the body fluid sample of interest.

The example housing 102 includes a body portion 116 that the sampling channel 106 extends through. The distal end of the body portion 116, at the outlet port 110, includes a connection means (not shown) that is used to couple a tube or the like to the outlet port 110. The collected body fluid sample exits out through the outlet port 110, which is in fluid communication with the tube, and travels through the connecting tube into a suitable container that collects and retains the body fluid sample for later testing.

The example housing 102 of the bodily-fluid diversion device 100 may optionally include finger grip tabs 118. The practitioner grasps the finger grip tabs 118 to hold the bodily-fluid diversion device 100 while the needle 104 is being used to puncture the patient, and to direct the needle 104 into the portion of the patient that has the body fluid of interest that is to be sampled. Any suitable finger grip or mechanical grip means may be used in the various embodiments. In some embodiments, the grip means may be configured for use by a robotic system.

Some embodiments of the bodily-fluid diversion device 100 may include an optional stop 120 at the proximal end of the housing 102. The stop 120 limits the travel distance of the needle 104 into the patient. Any suitable stop means may be used in the various embodiments.

A diversion chamber body portion 122, in the non-limiting example embodiment of FIG. 1, extends outwardly from the housing 102. The diversion chambers 112 are disposed within the diversion chamber body portion 122, and extend through the housing 102 to join with the sampling channel 106 at junction 124 located at a proximal end of the sampling channel 106. Accordingly, the diversion chambers 112 are in fluid communication with the sampling channel 106. The diversion chambers 112 are downstream of the inlet port 108 and are upstream of the outlet port 110. In some embodiments, the diameter of the diversion chambers 112 may be greater than the diameter of the inlet port 108 to facilitate flow of the first portion of the body fluid sample into the diversion chambers 112. For example, a predetermined amount of the first portion of the body fluid sample may range from 0.05 milliliter to 3 ml.

The non-limiting example embodiment of FIG. 1 illustrates seven diversion chambers 112. The diversion chambers 112 are located adjacent to each other and are downstream from a preceding diversion chamber 112. Here, the diversion chambers 112 cooperate to collect and retain the initial first portion of the body fluid. Any suitable number of diversion chambers 112, or even a single diversion chamber 112, may be used in alternative embodiments. Any suitable length and/or diameter may be used for the diversion chambers 112. Selection of the number of, length of, and/or diameter of the diversion chambers 112 may be made based on the amount of and/or characteristics of the initial portion of the body fluid sample that is acquired by the bodily-fluid diversion device 100. Further, the length of and/or diameter of individual diversion chambers 112 may vary between diversion chambers 112.

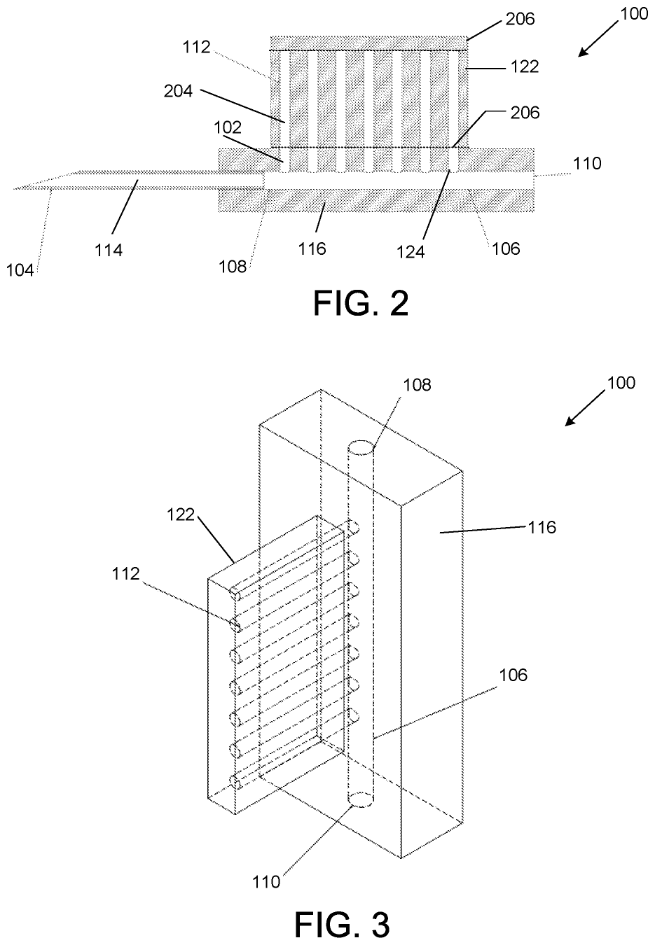

FIG. 2 is a cross-section view of an example embodiment of the bodily-fluid diversion device 100. When the bodily-fluid diversion device 100 is fabricated, the housing 102 and the diversion chamber body portion 122 may be separately formed. In an example embodiment, the sampling channel 106 may be drilled or the like into the housing 102. Alternatively, the housing 102 may be formed with the sampling channel 106 by an extrusion process or molding process.

A portion of the diversion chambers 112 may then be drilled into the housing 102 to a predefined depth into the housing 102 to establish the junctions 124 (denoted as chamber portion 202). A portion of the diversion chambers 112 (denoted as 204 in FIG. 2) may then be drilled into the diversion chamber body portion 122 to a predefined depth into the diversion chamber body portion 122.

Alternatively, the diversion chamber 112 portion 204 may be formed by drilling entirely through the diversion chamber body portion 122, wherein the holes at the distal end of diversion chamber body portion 122 may be sealed. For example, but not limited to, a plug may be inserted into each hole. Alternatively, a plate, sealing strip, or the like 206 may be secured over the distal end of the diversion chamber body portion 122 to close the holes.

In this example embodiment, chamber portions 202 of the diversion chambers 112 are formed into the housing 102, such as by drilling or the like. The location of each chamber portion 202 in the housing 102 corresponds to the location of each diversion chamber 112 portion 204 in the diversion chamber body portion 122.

Then, the housing 102 and the diversion chamber body portion 122 may be joined together so that the diversion chambers 112 and the diversion chamber body portion 122 align with each other and become fluidly coupled together. The outside surface of the housing 102 and a distal surface of the diversion chamber body portion 122 may be joined together using a securing means, such as a suitable adhesive, heat (melting), ultrasonic welding, or other fastening means such as screws, snaps or the like. An unexpected advantage of this embodiment is that a single housing 102 may be configured to be used with a plurality of different diversion chamber body portions 122 that have different numbers of diversion chambers 112, different lengths of diversion chambers 112, and/or different diameters of diversion chambers 112.

Alternatively, the housing 102 and the diversion chamber body portion 122 may be formed using a unibody construction wherein both are fabricated together as a single unibody piece. The unibody housing 102 and diversion chamber body portion 122 may be formed by machining, molding, extruding, or any other suitable manufacturing method. The sampling channel 106 and/or the diversion chamber(s) 112 may be formed into the unibody piece, such as by drilling or by using another suitable method.

FIG. 3 is an isometric view of an alternative embodiment of a bodily-fluid diversion device. In this example embodiment, the optional finger grip tabs 118 are omitted. This example embodiment may be configured to mate with an external device that is gripped by the practitioner and/or a machine, such as a robot device. Here, a single bodily-fluid diversion device 100 may be used with a particular gripping device that is particularly suited for obtaining the body fluid sample of interest. For example, the body fluid sample of interest may reside in the brain of the patient, and a robotic device may be used to precisely guide the needle 104 to a particular location in the patient's brain along a precisely defined path of travel. Here, it may be that the embodiment illustrated in FIG. 1, which is guided by hand by the practitioner who is grasping the finger grip tabs 118, is entirely unsuitable for this particular body fluid sampling. Rather, the embodiment illustrated in FIG. 3 may be coupled to a robotic arm so that the needle 104 can be precisely inserted into the patient.

Further, the example embodiment illustrated in FIG. 3 may be configured to receive different needles 104. Depending upon the particulars of the body fluid sampling process, different lengths and/or sizes of needles may be secured to the housing 102 as needed.

In some embodiments, a volume indicator may be used to sense the amount of the first portion of the body fluid sample that has come into the diversion chambers 112. For example, but not limited to, a clear transparent viewing window or the like may be used to permit the practitioner to view the amount of fluid that has come into the diversion chambers 112.

Alternatively, or additionally, a color sensitive material that changes color when coming into contact with the fluid may be used to indicate the fill level of the diversion chambers 112. The color sensitive material may be placed at one or more desired indicator locations along the length of the diversion chambers 112. Any suitable color-changing material that changes color whin in contact with a fluid may be used in the various embodiments.

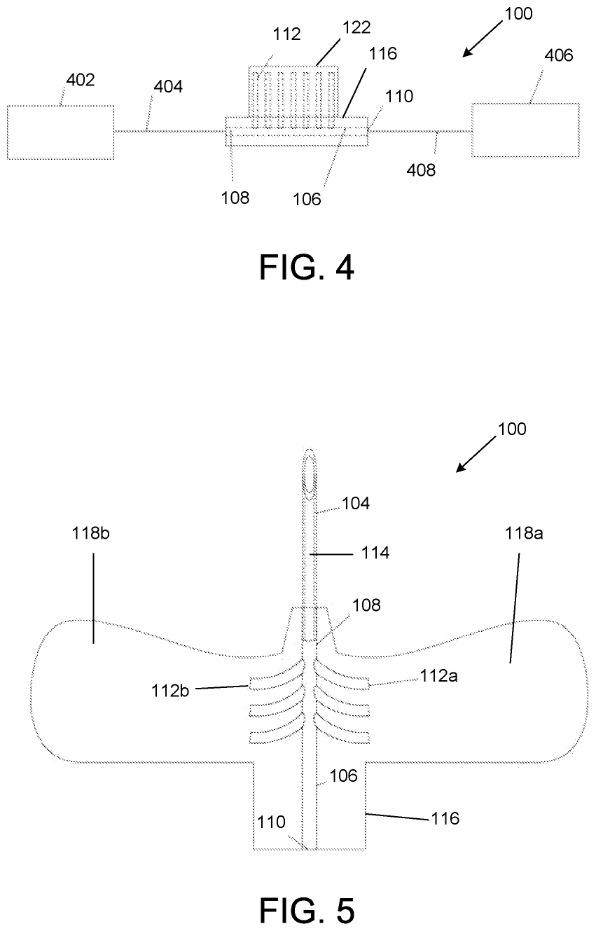

FIG. 4 is a block diagram of an alternative embodiment of the bodily-fluid diversion device 100. FIG. 4 illustrates an example embodiment wherein the primary bodily-fluid uptake device 402 is in fluid communication with the housing 102 via a tube 404 or the like that couples the primary bodily-fluid uptake device 402 to the housing 102 at the inlet port 108. In an example embodiment, the primary bodily-fluid uptake device 402 may be, but is not limited to, the needle 104. Any suitable primary bodily-fluid uptake device 402 now known or later developed are intended to be within the scope of this disclosure.

Optionally, the outlet port 110 of the housing 102 is fluidly coupled to a bodily-fluid collection device 406 via tube 408. The acquired later portion of the body fluid sample may be transferred to the bodily-fluid collection device 406 (wherein the initially acquired portion of the body fluid sample is retained within the diversion chambers 112).

The bodily-fluid collection device 406 may be configured to create a negative pressure (suction) within the sampling channel 106 to facilitate drawing of the second portion of the body fluid sample through the sampling channel 106 and out the outlet port 110. The practitioner may monitor the volume indicator to ensure that a sufficient amount of the first portion of the body fluid sample has been drawn into the diversion chambers 112. Then, the practitioner may actuate the bodily-fluid collection device 406 to generate the negative pressure. Since the fluid pressure at the junction location at the sampling channel 106 and the proximal end of the diversion chambers 112 are inherently the same, the first portion of the body fluid sample will remain, or will substantially remain, in the diversion chambers 112. (In some embodiments, a one-way valve at or near the proximal end of the diversion chambers 112 may be optionally used to prevent escape of any of the first portion of the body fluid sample from the diversion chambers 112.)

FIG. 5 is a cross-section view of an example embodiment of the bodily-fluid diversion device 100. In this example embodiment, the location of the diversion chambers 112 is within the two finger grip tabs 118. Here, a first plurality of diversion chambers 112a extend outward from the sampling channel 106 and into the first finger grip tab 118a, and an optional second plurality of diversion chambers 112b extend outward from the sampling channel 106 and into the second finger grip tab 118b in an opposing direction.

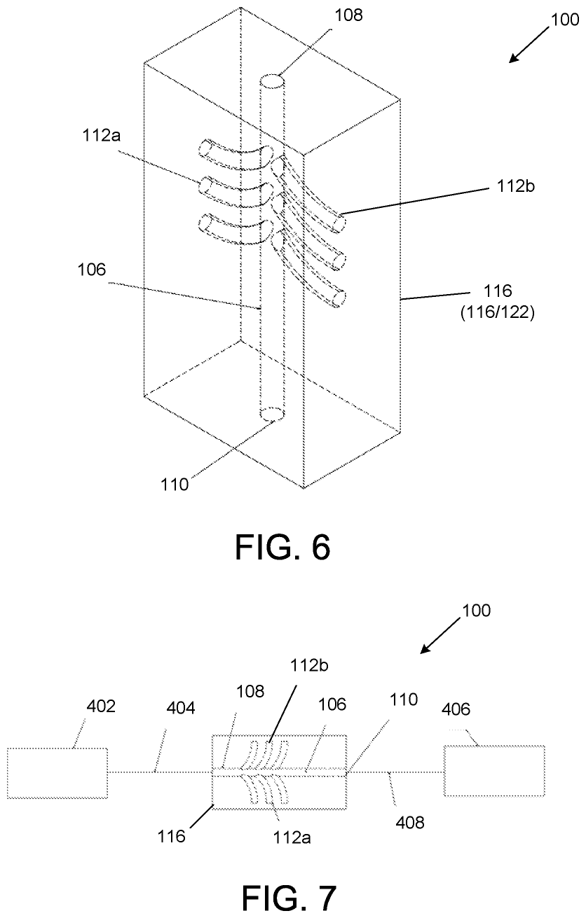

FIG. 6 is an isometric view of an alternative embodiment of the bodily-fluid diversion device 100 with two sets of diversion chambers 112a, 112b extending outward from the sampling channel 106 on two different sides and in two different directions. FIG. 7 is a block diagram of the alternative embodiment of the bodily-fluid diversion device 100 illustrated in FIG. 6. A first plurality of diversion chambers 112a and a second plurality of diversion chambers 112b are illustrated. In this embodiment, the diversion chambers 112a, 112b reside within the housing 102 (or equivalently, the housing 102 and the diversion chamber body portion 122 in a single unibody piece of material).

In some embodiments, a device such as a vacuum pump or the like may be used to partially evacuate the air from the diversion chambers 112 and sampling channel 106. A septum and/or a one-way valve may be used in proximity to the inlet port 108 and the outlet port 110 to maintain the partial vacuum and to receive a needle of the vacuum pump.

In one embodiment, a three-way valve may be used that has an opening pressure that is activated once the fluid pressure of the incoming body fluid sample is introduced at the inlet port 108. Accordingly, such valves remain closed after manufacture and transport of the bodily-fluid diversion device 100 to the use site. In some embodiments, the one-way or three-way valves may be manually opened by the practitioner, such as by actuating a switch, push button, or the like.

Additionally, the diversion chambers 112a, 112b are slightly curved. The diversion chambers 112a, 112b are optionally angled upwards from the axis of the sampling channel 106. An unexpected result of slightly curved and/or angled diversion chambers 112 is that the first portion of the acquired body fluid sample more readily flows into the curved diversion chambers 112. Further, the curved and/or angled diversion chambers 112 may require less lateral distance from the sampling channel 106 that the same length of straight diversion chambers 112. Accordingly, a smaller diversion chamber body portion 122 is required.

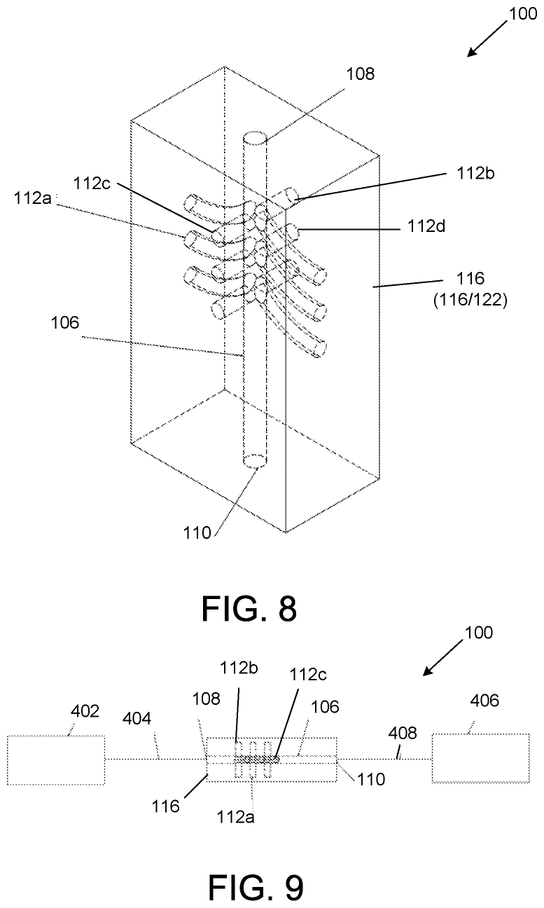

FIG. 8 is an isometric view of an alternative embodiment of the bodily-fluid diversion device 100 with multiple diversion chambers 112 extending outward from the sampling channel 106 in multiple different directions. FIG. 9 is a block diagram of the alternative embodiment of the bodily-fluid diversion device 100 illustrated in FIG. 8. A first plurality of diversion chambers 112a, a second plurality of diversion chambers 112b, a third plurality of diversion chambers 112c, and a fourth plurality of diversion chambers 112c are illustrated. In this embodiment, the diversion chambers 112a, 112b, 112c, 112d reside within the housing 102 (or equivalently, the housing 102 and the diversion chamber body portion 122 in a single unibody piece of material). Also, the diversion chambers 112 are illustrated for convenience as being curved.

In other embodiments, the diversion chambers 112 may be straight and/or angled at some degree with respect to the axis of the sampling channel 106. In some embodiments, the diameter of one or more of the diversion chambers 112 may decrease or increase along the length of the diversion chamber 112 to accommodate differing amounts of the first portion of the body fluid sample.

In other embodiments, the plurality of diversion chambers 112 may be arranged in any suitable arrangement within the diversion chamber body portion 122, and/or within the housing 102. In the various illustrated embodiments, the diversion chambers 112 may be aligned in a row, or in multiple rows, along an axis of the sampling channel 106. In some embodiments, the diversion chambers 112 may spiral about the sampling channel 106. Any suitable orientation of the diversion chambers 112 may be used in the various embodiments.

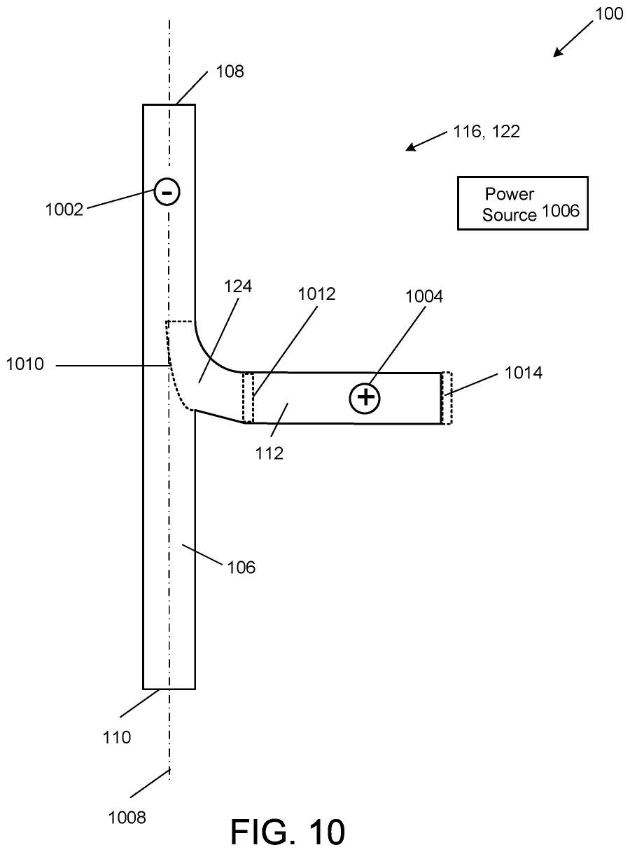

FIG. 10 is a cross-section view of an example alternative embodiment showing various optional features of the bodily-fluid diversion device 100. This example embodiment shows that an electrical charge may be used in order to facilitate the trapping of contaminant bacteria that have a negatively charged cell membrane. In this non-limiting example embodiment, the inlet port 108 is where the first portion bodily-fluid (contaminated) from the patient needle is transferred to. In one embodiment, a portion of the housing 102 at or proximate to the inlet port 108 may be optionally electrically charged with a negative charge 1002, and/or a negative charge 1002 may be applied (wherein the negatively charged cell membrane of the bacteria is repelled from the negative charged portion of the sampling channel), in order to drive the contaminate bacterial cells to the diversion chambers 112 after passing through the inlet port 108. Depending upon the embodiment, the negative charge 1002 may be located preceding the junction 124 and/or past the junction 124.

Further, a portion of the diversion chamber 112 may be optionally electrically charged with an electrical positive charge 1004 to attract negatively charged cell membrane of the bacteria. One skilled in the art appreciates that negatively charged cell membrane of the contaminate bacteria will be attracted by the positive electrical charge into a charged diversion chamber 112.

The electrical charges may be applied to a portion of the housing 102, the sampling channel 106, the diversion chamber body portion 122, and/or the diversion chambers 112 in a variety of manners by different embodiments. For example, electrodes, energized by an external or integrated power source 1006, may be used to apply the electrical charges.

Alternatively, or additionally, a charged coating may be applied in the diversion chambers 112 and/or the sampling channel 106 at desired locations. Alternatively, or additionally, a charged insertable sleeve or other portion may be inserted into the diversion chambers 112 and/or the sampling channel 106 at a desired location. Alternatively, or additionally, a material with an electrical charge may be embedded within the diversion chamber body portion 122 and/or the housing 102 at a desired location. For example, when the diversion chamber body portion 122 as a separately fabricated portion of the bodily-fluid diversion device 100, the diversion chamber body portion 122 may be made of a positively charged material. In some embodiments, a net positively charged polymer may be used to provide a positive charge to the diversion chambers 112. Additionally, or alternatively, a net negatively charged polymer may be used to provide a negative charge in the sampling channel 106 proximate to the junction 124.

In the embodiment illustrated in FIGS. 1-4, the plurality of diversion chambers 112 are arranged along a straight line axis extending perpendicularly outward from the axis of the sampling channel 106. In other illustrated embodiments, the diversion chambers 112 extend outward at a predefined angle from the axis of the sampling channel 106. Any suitable orientation of the diversion chambers 112 about the axis 1008 (FIG. 10) of the sampling channel 106 may be used in the various embodiments.

In some embodiments, the orientation of the diversion chambers 112 with respect to the sampling channel 106, during the process of acquiring the body fluid sample, may use the aid of gravity to draw and retain the first portion of the body fluid sample into the diversion chambers 112. Here, the orientation of the bodily-fluid diversion device 100 during the sampling process may be know and/or controlled. For example, the finger grip tabs 118 may be oriented is a manner that control the orientation of the housing 102 during the sampling process. When the orientation of the sampling channel 106 during the sampling process is known and/or controlled, the orientation of the diversion chambers 112 can be predefined to be in a downward direction during the sampling process. Accordingly, gravity will help draw and retain the first portion of the body fluid sample into the diversion chambers 112.

Surface modifiers may be introduced to any of the surfaces of the bodily-fluid diversion device 100 in order to aid in the flow of the body fluid sample. For example, but not limited to, surface modifiers that make surfaces hydrophilic may be introduced to the diversion chambers 112 in order to capture the first portion of body fluid sample. Alternatively, or additionally, surface modifiers may be applied to other portions of the bodily-fluid diversion device 100 in order to drive bodily-fluid flow. Surface modifiers may be applied or integrated into the bodily-fluid diversion device 100 through coatings, insert molding, adhesive bonding, ultrasonic welding, or the like. For example, but not limited to, an insert molding of the diversion chambers 112 may be used for the integration of a hydrophilic polymer into the diversion chambers 112 in order to enhance capillary action. In yet another embodiment, metal or glass 1010 may be insert molded into the diversion chambers 112 and/or proximate to junction 124 in order to enhance capillary action

In some embodiments, a one-way valve 1012 may be disposed within the diversion chambers 112 at or proximate to the junction 124. The one-way valve 1012 may open to permit flow of the first portion of the body fluid sample into the diversion chambers 112. When the diversion chamber 112 is full, or substantially full, of the first portion of the body fluid sample such that flow ceases into the diversion chamber 112, back flow pressure closes the one-way valve 1012 so that the accumulated first portion of the body fluid sample does not leave the diversion chamber 112. Such a one-way valve 1012 may be used with any embodiment described herein. Any suitable one-way valve 1012 may be used in the various embodiments without departing from the scope of this disclosure.

In other embodiments, other processes and apparatus may be used to attract the first portion of the body fluid sample into the diversion chambers 112. A substantial pressure equalization between the sampling channel 106 and the diversion chambers 112 (since a higher pressure within the diversion chambers 112 would retard flow of the first portion of the body fluid sample into the diversion chambers 112).

Alternatively, a negative pressure in the diversion chambers 112, with respect to the initial atmospheric air pressure within the sampling channel 106, may be used by some embodiments (since a lower pressure within the diversion chambers 112 would facilitate flow of the first portion of the body fluid sample into the diversion chambers 112).

As another example, in a closed system, the bodily-fluid collection device 406 (FIG. 4) may be configured to create a negative pressure (suction) within the sampling channel 106. Such a negative pressure facilitates the drawing of the second portion of the body fluid sample through the sampling channel 106 and out the outlet port 110.

In an example embodiment, capillary action may be alternatively, or additionally used, to draw the first portion of the body fluid sample into the diversion chambers 112. Here, the relatively small diameter of the diversion chambers 112 facilitates entry of the first portion of the body fluid sample into the diversion chambers 112 due to the surface tension of the liquid body fluid sample in proximity to the junction 124, and in combination with adhesive forces between the liquid body fluid sample and the walls of the diversion chambers 112 that propels the liquid body fluid sample into the diversion chambers 112.

In some embodiments, an air permeable fluid barrier 1014 (interchangeably referred to herein as a filter) may be disposed within the diversion chambers 112 at, or proximate to, the distal end of the diversion chambers 112 when such diversion chambers 112 have an exit into ambient surroundings. Here, one skilled in the art appreciates that air initially resides in the interior of the diversion chambers 112. As the first portion of the body fluid sample enters into the diversion chambers 112, a buildup of increasing air pressure occurs at the distal end of the diversion chamber 112. This pressure increase retards later flow of the first portion of the body fluid sample into the diversion chambers 112. Accordingly, the air permeable fluid barrier 1014 allows air to escape from the interior of the diversion chamber 112 so that air pressure does not build up within the diversion chamber 112. When and if the first portion of the body fluid sample entering into the diversion chamber 112 arrives at the distal end of the diversion chamber 112, the air permeable fluid barrier 1014, which is liquid impermeable, prevents outflow of the body fluid sample from the exit of the diversion chamber 112. Accordingly, since the diversion chamber 112 is full of the first portion of the body fluid sample, the second portion of the body fluid sample may proceed down the sampling channel 106 for collection. Such an air permeable fluid barrier 1014 may be used with any embodiment described herein. Any suitable air permeable fluid barrier 1014 may be used in the various embodiments without departing from the scope of this disclosure.

Alternatively, or additionally, a one-way valve 1012 may be similarly disposed at the distal end of a diversion chamber 112 that remains open to permit flow of air out from the diversion chambers 112. When the diversion chamber 112 is full, or substantially full (with the first portion of the body fluid sample plus the pressurized air at the distal end of the diversion chamber 112), the one-way valve 1012 closes by a slight back flow of the first portion of the body fluid sample from the diversion chamber 112 so that the accumulated first portion of the body fluid sample does not leave the diversion chamber 112. Such a one-way valve 1012 may be used with any embodiment described herein. Any suitable one-way valve 1012 may be used in the various embodiments without departing from the scope of this disclosure.

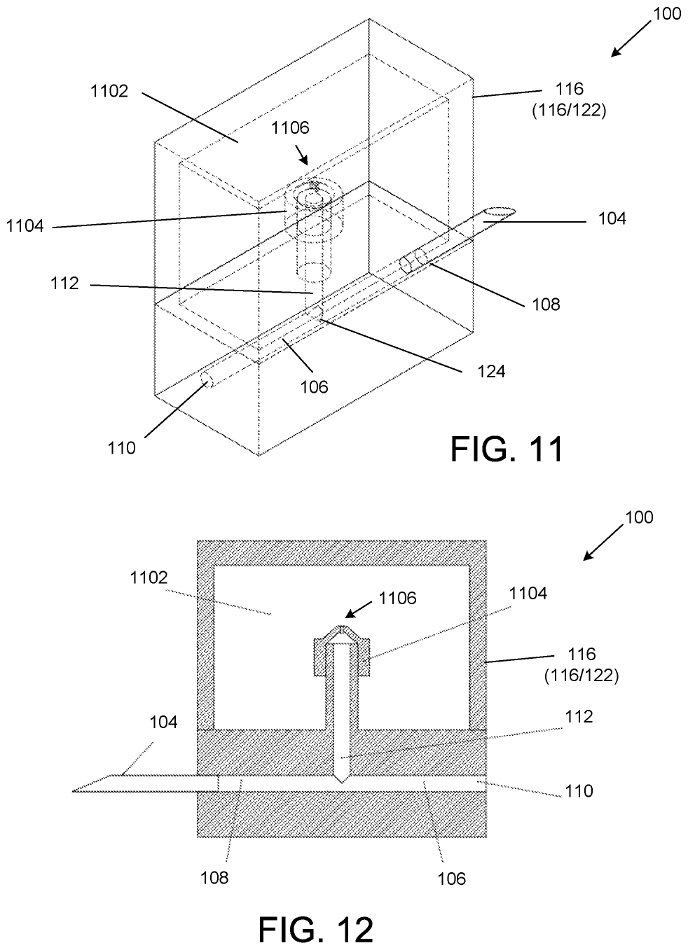

FIG. 11 is an isometric view of an example embodiment of a bodily-fluid diversion device 100 with an air tank 1102. FIG. 12 is a cross-section view of the example embodiment of the bodily-fluid diversion device 100 with the air tank 1102. FIG. 13 is a block diagram of an alternative embodiment of the bodily-fluid diversion device 100 with the air tank 1102. The embodiment illustrated in FIG. 13 functions similarly to the above-described embodiments illustrated in FIGS. 4, 7, and 9, and is not described herein for brevity. Further, the example bodily-fluid diversion device 100 illustrated in FIGS. 11-13 illustrate a single diversion chamber 112. Alternative embodiments may have a plurality of diversion chambers 112.

Prior to use, the diversion chamber 112 is filled with air at atmospheric pressure. The distal end of the diversion chamber 112 is fluidly coupled to the air tank 1102.

With an air tank 1102, as the initial first portion of the body fluid enters into the diversion chamber 112, the air in the diversion chamber 112 exits into the air tank 1102, thereby avoiding (or at least significantly reducing) the buildup of air pressure in the diversion chamber 112. By preventing air pressure buildup in the diversion chamber 112 (by allowing air to escape the diversion chamber 112 into the air tank 1102), a desired amount of the initial first portion of the body fluid can be collected into and be retained within the diversion chamber(s) 112. In the various embodiment, the size and dimensions of the air tank 1102 may be defined so that when the desired amount of the initial first portion of the body fluid has been acquired into the diversion chamber(s) 112, air pressure equals or substantially equals the fluid pressure such that the incoming subsequent second portion of the body fluid passes through the sampling channel 106 for collection. In embodiments with a one-way valve, the one-way valve may be configured to close when the air/fluid pressure equals or substantially equals the closing pressure threshold of the one-way valve. In some embodiments, the air tank 1102 may be configured to allow a piston and spring to regulate pressure within the air tank 1102. In other embodiments, a one-way valve with a sensor may close at a given pressure difference (and/or backflow) across the one-way valve so that the one-way valve may close before the pressure is equal or substantially equal.

In this example embodiment, an optional diversion chamber cap 1104 over the distal end of the diversion chamber 112 permits the air to escape from the diversion chamber 112 into the air tank 1102. When the diversion chamber 112 has filled with the initial first portion of the body fluid, the diversion chamber cap 1104 prevents flow of the initial first portion of the body fluid into the air tank 1102. Here, in one embodiment, an orifice 1106 disposed in the diversion chamber cap 1104 permits a limited amount of air to escape the diversion chamber 112 while preventing (or at least retarding) flow of the initial first portion of the body fluid into the air tank 1102.

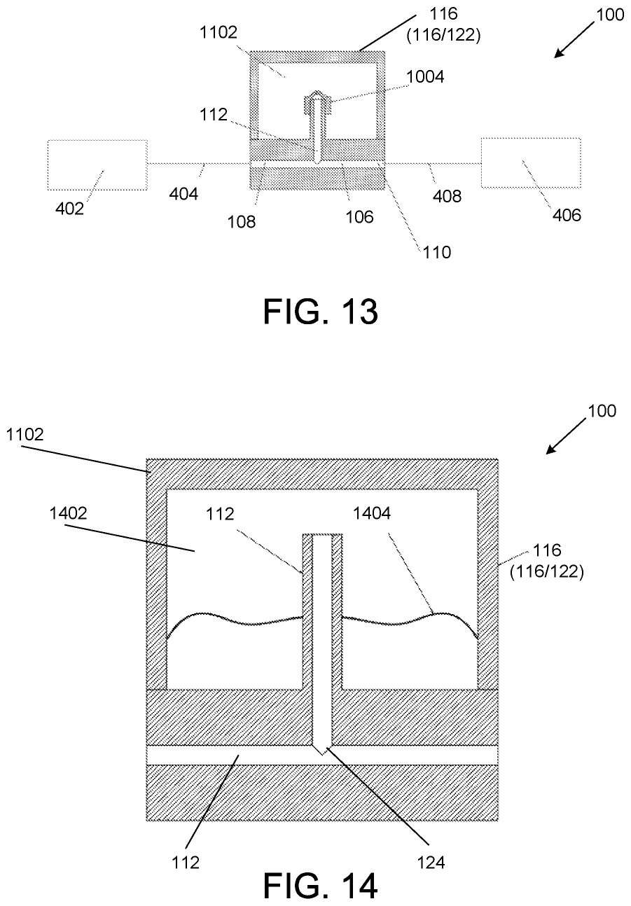

FIG. 14 is a cross-section view of an example embodiment of a bodily-fluid diversion device 100 with an air tank 1102. Here, the optional diversion chamber cap 1104 is omitted. As the initial first portion of the body fluid is received, the received initial first portion of the body fluid flows through the diversion chamber 112 and exits out of the distal end of the diversion chamber 112 to enter into the air tank 1102. The interior volume 1402 of the air tank 1102 acts as a collection portion so that a greater amount of the initial first portion of the body fluid 1404 can be acquired before the subsequent second portion of the body fluid is collected. As contrasted with the embodiment illustrated in FIGS. 11-13, a greater amount of the initial first portion of the body fluid can be acquired when the diversion chamber cap 1104 is omitted, thereby increasing the likelihood that all contaminants are acquired in the initial first portion of the body fluid. If a lesser amount of the initial first portion of the body fluid is required while ensuring that the subsequent second portion of the body fluid is contaminant free, the diversion chamber cap 1104 can be used to limit the amount of the acquired initial first portion of the body fluid.

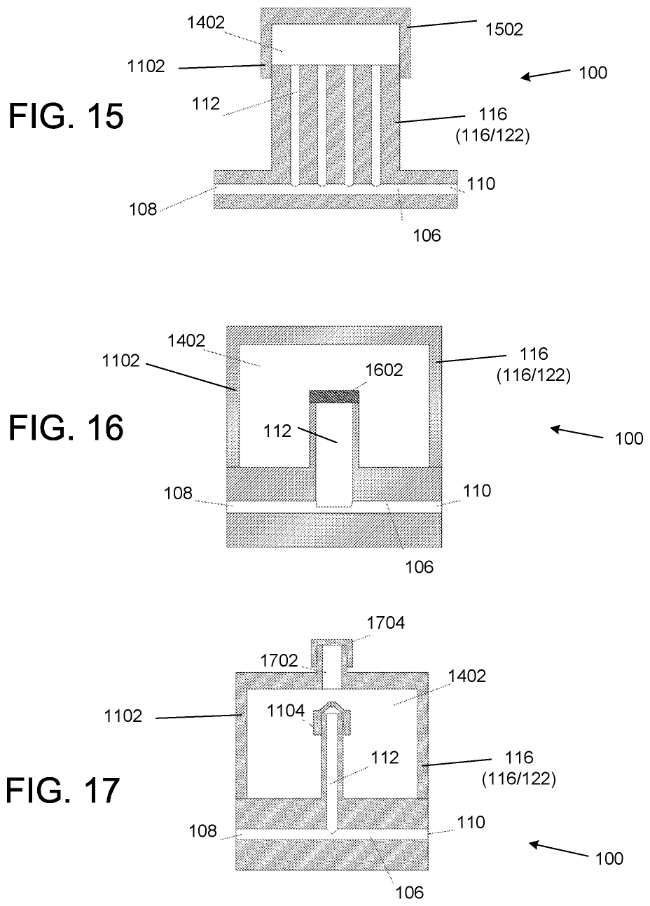

FIG. 15 is a cross-section view of an example embodiment of a bodily-fluid diversion device 100 with an air tank 1102 (and no diversion chamber cap 1104). In this non-limiting example embodiment, a plurality of diversion chambers 112 each have their end in fluid communication with the air tank 1102. In this example embodiment, the diversion chamber cap 1104 may be formed as a suitable cap-like structure that is secured to the diversion chamber body portion 122 after the diversion chamber body portion 122 has been fabricated. Here, in the illustrated embodiment, part of the initial first portion of the body fluid is free to flow into the air tank 1102. In alternative embodiments, the optional diversion chamber cap 1104, a filter, a one-way valve or other means may be used proximate to the distal end of the diversion chambers 112 to prevent flow of the initial first portion of the body fluid into the air tank 1102.

FIG. 16 is a cross-section view of an example embodiment of a bodily-fluid diversion device with an air tank and a filter 1602 at a distal end of the diversion chamber 112. The filter 1602 is air permeable and liquid impermeable. As the initial first portion of the body fluid begins to enter into the diversion chamber(s) 112, the air in the diversion chamber 112 is expelled into the air tank 1102. When the initial first portion of the body fluid reaches the distal end of the diversion chamber 112, the filter 1602 prevents the initial first portion of the body fluid from escaping the diversion chamber 112 and entering into the air tank 1102. In one example embodiment, the filter 1602 may be made of a cellulose material, such as but not limited to, Carbo methylcellulose, that absorbs liquid and swells to from a filter seal.

FIG. 17 is a cross-section view of an example embodiment of a bodily-fluid diversion device 100 with an air tank having an air outlet 1702. As air is expelled from the diversion chamber 112 into the air tank 1102, air pressure begins to build up within the air tank 1102. In some embodiments, it may be desirable to limit air pressure buildup within the air tank 1102. The air outlet 1702 may be configured to allow pressured air in the air tank 1102 to escape into the ambient region having atmospheric pressure air. An optional air tank vent cap 1704 may be used to control the air pressure at which the air within the air tank 1102 is permitted to vent out from the air tank 1102 into the ambient region.

It should be emphasized that the above-described embodiments of the bodily-fluid diversion device 100 are merely possible examples of implementations of the invention. Many variations and modifications may be made to the above-described embodiments. All such modifications and variations are intended to be included herein within the scope of this disclosure and protected by the following claims.

Furthermore, the disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower, or equal in scope to the original claims, are to be considered within the subject matter of the inventions described herein.

Therefore, having thus described the invention, at least the following is claimed:

1. A bodily-fluid diversion device, comprising:
a housing,
a sampling channel disposed within and extending through the housing,
wherein the sampling channel is defined by an inlet port at a proximate end of the housing and an outlet port at a distal end of the housing,
wherein the inlet port is fluidly coupled a proximal end of a needle having a distal end configured to penetrate a patient to acquire a body fluid sample,
wherein the sampling channel extends between the inlet port and the outlet port and extends through the housing, and
wherein the sampling channel receives and transfers portions of the body fluid sample received from the needle from the inlet port to the outlet port of the housing;
a diversion chamber body portion directly secured to the housing; and
a diversion chamber disposed within the diversion chamber body portion,
wherein the diversion chamber extends from a proximal end to a distal end,
wherein the proximal end of the diversion chamber is in fluid communication with the sampling channel, and
wherein the distal end of the diversion chamber terminates before an exterior end of the diversion chamber body portion,
wherein an initial first portion of the body fluid received from the needle at the inlet port of the housing is transferred through the sampling channel inlet port and into the diversion chamber,
wherein, after the diversion chamber is filled with the initial first portion of the body fluid, a subsequent second portion of the body fluid received from the needle is communicated through the sampling channel to the outlet port while the initially received first portion of the body fluid is retained in the diversion chamber, and
wherein the subsequently received second portion of the body fluid exits the outlet port for collection into a collection device while the initially received first portion of the body fluid is retained in the diversion chamber.

2. The bodily-fluid diversion device of claim 1, further comprising:
a one-way valve disposed within the diversion chamber,
wherein the one-way valve is proximate to a junction where the proximal end of the diversion chamber joins with the diversion chamber,
wherein the initially received first portion of the body fluid passes through the one-way valve into the diversion chamber, and
wherein the one-way valve, after the diversion chamber is filled with the initially received first portion of the body fluid, closes to prevent the initially received first portion of the body fluid collected in the diversion chamber from exiting the diversion chamber as the subsequently received second portion of the body fluid is passing through the sampling channel.

3. The bodily-fluid diversion device of claim 1, further comprising:
a surface modification disposed within at least one of the sampling channel and the diversion chamber proximate to a junction where the proximal end of the diversion chamber joins with the diversion chamber,
wherein the surface modification facilitates communication through the sampling channel and into the diversion chamber.

4. The bodily-fluid diversion device of claim 1, wherein the diversion chamber is a first diversion chamber, and further comprising:
a second diversion chamber having a proximal end in fluid communication with the sampling channel,
wherein the first diversion chamber and the second diversion chamber cooperate to collect and retain the initial first portion of the body fluid.

5. The bodily-fluid diversion device of claim 4, wherein the second diversion chamber is located adjacent to and downstream from the first diversion chamber, and wherein the second diversion chamber has a length and diameter that is the same as a length and a diameter of the first diversion chamber.

6. The bodily-fluid diversion device of claim 1,
wherein the diversion chamber is a first one of a plurality of diversion chambers each having a proximal end in fluid communication with the sampling channel,
wherein the plurality of diversion chambers are located adjacent to each other and are arranged downstream from a preceding diversion chamber, and
wherein the plurality of diversion chambers cooperatively collect and retain the initial first portion of the body fluid.

7. The bodily-fluid diversion device of claim 1, further comprising:
an air permeable and liquid impermeable barrier located at the distal end of the diversion chamber,
wherein air initially occupying the diversion chamber exits through the air permeable and liquid impermeable barrier as the initial first portion of the body fluid is collected into the diversion chamber, and
wherein the air permeable and liquid impermeable barrier is impermeable to the initial first portion of the body fluid.

8. The bodily-fluid diversion device of claim 1, further comprising:
an air tank located proximate to the distal end of the diversion chamber,
wherein air initially residing within the diversion chamber is displaced by the initially received first portion of the body fluid, and
wherein the displaced air exits the diversion chamber and enters into the air tank.

9. The bodily-fluid diversion device of claim 1, further comprising:
an air permeable and liquid impermeable barrier located at the distal end of the diversion chamber,
wherein air initially occupying the diversion chamber exits through the air permeable and liquid impermeable barrier and into the air tank as the initial first portion of the body fluid is collected into the diversion chamber, and
wherein the air permeable and liquid impermeable barrier is impermeable to the initial first portion of the body fluid.

10. The bodily-fluid diversion device of claim 1, further comprising:
a one-way valve located in the sampling channel,
wherein the subsequently received second portion of the body fluid passes through the one-way valve and out through the outlet port of the housing, and
wherein the one-way valve prevents the subsequently received second portion of the body fluid collected in the sampling channel from exiting back through the inlet port of the housing.

11. The bodily-fluid diversion device of claim 1, wherein the diversion chamber is positively charged to attract bacterial having a negatively charged cell membrane.

12. The bodily-fluid diversion device of claim 11, wherein the diversion chamber comprises:
a positively charged polymer coating disposed along a portion of the diversion chamber.

13. The bodily-fluid diversion device of claim 11, wherein the diversion chamber comprises:
a positively charged insert molding that is inserted into a portion of the diversion chamber.

14. The bodily-fluid diversion device of claim 11, wherein a positive charge is applied to the diversion chamber by an electrical charging device.

15. The bodily-fluid diversion device of claim 11, wherein a portion of the sampling channel preceding a juncture between the diversion chamber and the sampling channel is negatively charged, wherein the negatively charged cell membrane of the bacteria is repelled from the negative charged portion of the sampling channel.

16. The bodily-fluid diversion device of claim 1, wherein the initial first portion of the body fluid is drawn into the diversion chamber by capillary action.

17. The bodily-fluid diversion device of claim 1, wherein the diversion chamber is disposed in the diversion chamber body portion extending perpendicularly outward from the sampling channel.

18. The bodily-fluid diversion device of claim 1,
wherein the diversion chamber body portion is secured to the housing after the diversion chamber has been disposed into the diversion chamber body portion, and
wherein a distal surface of the diversion chamber body portion is secured to an outside surface of the housing using a securing means.

19. The bodily-fluid diversion device of claim 18,
wherein the diversion chamber is drilled into the diversion chamber body portion prior to securing the diversion chamber body portion to the housing.

20. The bodily-fluid diversion device of claim 1,
wherein the diversion chamber body portion and the housing are fabricated together as a single unibody piece.
